(19) United States
(12) Patent Application Publication
(10) Pub. No.: US 2021/0178030 A1
(43) Pub. Date: Jun. 17, 2021

(54) Efficient Carrying Portable Constant Negative Pressure Suction Apparatus for Outdoor Use

(71) Applicant: Jinfang XIAO, Guangzhou, Guangdong (CN)

(72) Inventor: Jinfang XIAO, Guangzhou, Guangdong (CN)

(21) Appl. No.: 16/758,612

(22) PCT Filed: Nov. 10, 2017
(86) PCT No.: PCT/CN2017/110505

§ 371 Date: Apr. 23, 2020

(30) Foreign Application Priority Data
Oct. 24, 2017 (CN) 201711007233.4

(51) Int. Cl. A61M 1/00

(52) U.S. Cl. CPC A61M 1/0031; A61M 1/008; A61M 2205/3331; A61M 2205/0216; A61M 1/005

(57) ABSTRACT

The present efficient support portable constant negative pressure suction apparatus for outdoor use includes: a negative pressure suction ball extending from the proximal end to the distal end, the negative pressure suction ball being provided with an internal sac structure; the internal sac structure includes: a round gas exhaust valve, a spherical sac, and an anti-reverse flow flap arranged in sequence from the proximal end to the distal end; and also including: a negative pressure suction tube, the negative pressure suction tube being in communication with the negative pressure suction ball and the negative pressure suction tube being provided with a suction control channel.

The present application claims the priority of Chinese Patent Application No. 2017110072334 filed with the Chinese Patent Office on Oct. 24, 2017 and entitled “Efficient Carrying Portable Constant Negative Pressure Suction Apparatus for Outdoor Use”, the entire content of which is incorporated herein by reference.

### TECHNICAL FIELD

The present disclosure relates to the technical field of medical equipment for both military and civilian purposes, in particular to an efficient-carrying (i.e., “efficient-support”, the term “efficient-support” means that the suction device of the present disclosure can be efficiently carried into the human body, for example, into the respiratory tract, esophagus and stomach) portable constant-negative-pressure suction device for outdoor/field use (an efficient carrying portable constant negative pressure suction apparatus for outdoor use).

### BACKGROUND

In a regular medical field, negative-pressure suction relies on negative-pressure suction effect generated by a central-negative-pressure suction center to suck sputum, blood bleeding at wound and other secretions or exudates. Due to the central negative pressure, it requires large equipment investment and high requirements. For example, a vacuum suction station generally comprises two water ring vacuum pumps, a vacuum tank, solenoid valves, water-gas separators, electrical control boxes, connecting pipelines, butterfly valves and shut-off valves, which are all installed in a suction pump room. Regarding suction pipelines: a main pipe and branch pipes are galvanized pipes, after entering a ward, a copper pipe is used, fixed onto an integrated equipment cover, and connected to a gas terminal valve (gas head). Regarding a gas terminal connector (plug-in quick connector): it is normally in a sealed state, wherein when a suction bottle is inserted, a gas terminal automatically opens, generating negative pressure. The suction device (bottle) is an independent device with a volume of 1-5 L. When in use, the suction bottle is inserted into a corresponding insertion orifice, so as to generate a negative pressure in the bottle, thereby smoothly sucking out dirt in the body of a patient. The indicating value of the vacuum gauge on the vacuum pump should be adjusted within a range of −0.03 Mpa to −0.07 Mpa after installation, which may meet use requirements for the ward. When the indicating value is lower than −0.03 Mpa, the vacuum pump automatically starts pumping, and when the pointer reaches −0.07 Mpa, it automatically stops pumping. A solenoid valve is installed on a gas intake pipeline of the pump, and the solenoid valve and a pump motor are started synchronously, that is, when the motor is started, the solenoid valve is automatically opened and connected to the vacuum system; when the pump stops working, the solenoid valve is automatically closed to prevent, when the pumping is stopped, water and air from flowing back into the vacuum system. Moreover, the system uses two water ring vacuum pumps which are alternately used. That is, one pump works first after the power is turned on, and automatically stops after reaching a certain negative pressure value (−0.07 Mpa), and when the negative pressure of the vacuum system rises to −0.03 Mpa, another vacuum pump is automatically started, which steps are repeated so that the system is kept to be within a set vacuum range. This is basic equipment of a central hospital. This kind of negative pressure equipment is commonly used, and obtained data range, −0.03 Mpa to −0.07 Mpa, is a result of big data. The negative-pressure suction device has been clinically applied to patients for more than 100 billion times. Simple electric suction devices are often used in medical aid stations, which has a relatively large volume, and is inconvenient to carry, resulting in relatively complex field operations.

At present, existing manual or foot-controlled suction devices cannot meet outdoor/field suction requirements because the negative pressure for suction cannot achieve pressure requirement and the gas leakage rate is high, and there is no suction devices suitable for intra-airway bleeding or hematocele caused by chest penetrating injuries on the field battlefield, or drowning, or auxiliary suction devices required when the elderly at home are too weak to spit sputum. The existing portable negative-pressure suction device can only perform cleaning of blood, sputum or secretions in the oral cavity, but cannot clean blood, sputum or secretions in the deep nasal cavity, pharyngeal cavity, trachea, bronchus, stomach.

In addition, when carrying or entering a first scene or rescuing and salvaging patients or the wounded in a first-line battlefield, the suction device is very inconvenient to carry and the operation thereof is complicated. Studies have shown that the suffocation death rate of patients or the wounded is 15.7%, with the death caused by blocking the airway by airway foreign bodies, blood, sputum, or secretions. In field battlefields and training grounds, airway injuries and wound bleeding require a suction device suitable for the field. Especially, the elderly have poor cough reflex ability, expectoration powerless, and high incidence of airway blocking by airway secretions or sputum or blood at home or a place away from the medical place, which may easily cause asphyxia hypoxia, hypoxic encephalopathy or death, so it is extremely necessary to prepare a disposable sputum suction device at home. Even if the hospital has sputum suction devices, it takes a long time to prepare in an emergency. If a handy disposable suction device is prepared by a bed or in a first aid box, further rescue opportunities may be obtained. When rescuing a drowned person at a first scene in the field, who sucked much water in the airway and stomach, suction devices suitable for the field are also needed.

Therefore, it has become a technical problem that urgently needs to be solved by a person skilled in the art, about how to provide an efficient-support portable constant-negative-pressure suction device for outdoor use that can satisfy the stable negative-pressure suction pressure in the range of −0.03 Mpa to −0.07 Mpa, has a calculated negative pressure range of −0.3099 kgf/cm² to −0.702 kgf/cm², and simultaneously can be conveniently carried and operated.

### SUMMARY

Objects of the present disclosure are to provide an efficient-support portable constant-negative-pressure suction device for outdoor use, so as to solve the technical problems of high gas leakage rate, unstable negative pressure and inconvenient carrying caused by that existing negative-pressure suction device cannot reach an operation negative pressure of −0.03 Mpa to −0.07 Mpa.

The present disclosure provides an efficient-support portable constant-negative-pressure suction device for outdoor use, including: a negative-pressure suction ball extending from a proximal end to a distal end, wherein the negative-pressure suction ball is in an internal cystic structure, and the internal cystic structure includes a circular gas exhaust valve, a balloon and an anti-backflow valve which are sequentially arranged from the proximal end to the distal end; and the device further comprises a negative-pressure suction tube, wherein the negative-pressure suction tube communicates with the negative-pressure suction ball, and the negative-pressure suction tube has a suction control channel.

Optionally, a support member is provided on the inner wall of the balloon of the negative-pressure suction ball, so that the support member can resist the squeezing of balloon wall toward the center line of the negative-pressure suction ball, to return to a spherical state. Optionally, the support member on the inner wall of the balloon of the negative-pressure suction ball is in a soft structure.

Optionally, a longitudinal elastic plate and a lateral elastic wire are further provided on the inner wall of the balloon of the negative-pressure suction ball, and the support member, the longitudinal elastic plate and the lateral elastic wire together resist the squeezing of balloon wall toward the center line of the negative-pressure suction ball, so as to return to the spherical state.

Optionally, the longitudinal elastic plate and the lateral elastic wire are fitted in the negative-pressure suction ball, and the longitudinal elastic plate includes at least two ridge plates, and the lateral elastic wire includes at least two lateral elastic wires, which are alternate in pairs and combined with the longitudinal elastic plate. Optionally, the longitudinal elastic plate includes at least two ridge plates provided opposite to each other, the lateral elastic wire includes at least two lateral elastic wires, wherein two ends of each lateral elastic wire are respectively connected to the two opposite ridge plates, so that the lateral elastic wire is cooperated with the longitudinal elastic plate.

Optionally, when the balloon of the negative-pressure suction ball is squeezed, the circular gas exhaust valve protrudes to exhaust gas, and the anti-backflow valve closes the negative-pressure suction tube; when the squeezing is released, the circular gas exhaust valve is retracted, the anti-backflow valve is opened; and when the balloon of the negative-pressure suction ball is in an expanded state, the balloon of the negative-pressure suction ball has a circular section and is elliptical as a whole.

Compared with the prior art, the efficient-support portable constant-negative-pressure suction device for outdoor use of the present disclosure has following advantages:

the efficient-support portable constant-negative-pressure suction device for outdoor use provided by the present disclosure, when used outside hospitals, especially when used for rescuing the wounded in the field and battlefield or in places away from negative-pressure suction areas, can effectively solve the problems of airway obstruction caused by airway sputum and blood of the wounded and the sick, and especially prevent dyspnea, suffocation, hypoxia and even life-threatening cases of patients occurring due to chest puncture injury-caused bleeding or drowning-caused emergency airway conditions. Moreover, the efficient-support portable constant-negative-pressure suction device for outdoor use provided by the present disclosure has extremely convenient operation, and is simple, practical, scientific and safe, which can be used by both professional or non-professional persons after basic training, and can be also prepared for family elderly, which can effectively fill the huge gaps in the demand of future battlefield and market.

The present disclosure also provides an efficient-support portable constant-negative-pressure suction device for outdoor use, including: a movable negative-pressure gas exhaust valve extending from a proximal end to a distal end, a negative-pressure suction ball and a negative-pressure suction tube, wherein the negative-pressure gas exhaust valve and the negative-pressure suction ball communicate with the negative-pressure suction tube through a suction control channel.

Optionally, the negative-pressure gas exhaust valve has a manual knob, the negative-pressure gas exhaust valve is provided with a knob port channel and a drain hole, and the manual knob can be taken out from the knob port channel and the drain hole.

Optionally, the balloon of the negative-pressure suction ball has a balloon cavity, and the balloon cavity communicates with a negative-pressure oxygen suction tube.

Optionally, a support spring is provided inside the balloon of the negative-pressure suction ball; or a support spring and an anti-backflow valve are provided inside the balloon of the negative-pressure suction ball.

Optionally, the inner tube wall of the negative-pressure suction tube is provided with at least one internal rifling.

Optionally, the negative-pressure suction tube has a suction control hole, and the hole wall of the suction control hole is provided with at least one internal groove line.

This efficient-support portable constant-negative-pressure suction device for outdoor use has the same advantages over the prior art as the above-mentioned efficient-support portable constant-negative-pressure suction device for outdoor use, which will not be repeated here.

### BRIEF DESCRIPTION OF DRAWINGS

In order to more clearly illustrate technical solutions of specific embodiments of the efficient-support portable constant-negative-pressure suction device for outdoor use of the present disclosure or of the prior art, drawings which are needed for use in description of the specific embodiments or the prior art will be introduced briefly below. Apparently, following described drawings are some special embodiments of the present disclosure, and a person ordinarily skilled in the art can also obtain other drawings according to these drawings, without paying inventive efforts, and the other undescribed drawings all fall within the scope of protection of the present disclosure.

FIG. 1 is a schematic perspective view showing a structure of an efficient-support portable constant-negative-pressure suction device for outdoor use provided in embodiments of the present disclosure when expanded in a resting state;

FIG. 2 is a schematic perspective view showing a structure of an efficient-support portable constant-negative-pressure suction device for outdoor use provided in embodiments of the present disclosure;

FIG. 3 is a schematic perspective view showing a structure of an efficient-support portable constant-negative-pressure suction device for outdoor use provided in embodiments of the present disclosure, which is in a pressurized state;

FIG. 4 is a schematic perspective view showing a structure of a negative-pressure gas exhaust valve of the efficient-support portable constant-negative-pressure suction device for outdoor use provided in embodiments of the present disclosure;

FIG. 5 is a schematic perspective view showing a structure of a gas exhaust structure of the negative-pressure gas exhaust valve of the efficient-support portable constant-negative-pressure suction device for outdoor use provided in embodiments of the present disclosure;

FIG. 6 is a schematic perspective view showing a structure of the efficient-support portable constant-negative-pressure suction device for outdoor use provided in embodiments of the present disclosure, which is in a first resting state in a single operation;

FIG. 7 is a schematic perspective view showing a structure of the efficient-support portable constant-negative-pressure suction device for outdoor use provided in embodiments of the present disclosure, which is in a second resting state in a single operation;

FIG. 8 is a schematic perspective view showing a structure of a spring frame of the efficient-support portable constant-negative-pressure suction device for outdoor use provided in embodiments of the present disclosure, when resting;

FIG. 9 is a schematic perspective view showing a structure of the spring frame of the efficient-support portable constant-negative-pressure suction device for outdoor use provided in embodiments of the present disclosure, when pressurized;

FIG. 10 is a schematic perspective view showing a structure of the efficient-support portable constant-negative-pressure suction device for outdoor use provided in embodiments of the present disclosure, which is in a third resting state in a single operation;

FIG. 11 is a schematic perspective view showing a structure of the efficient-support portable constant-negative-pressure suction device for outdoor use provided in embodiments of the present disclosure, which is in a pressurized state in a single operation;

FIG. 12 is a schematic perspective view showing a structure of a negative-pressure suction tube with internal riflings, of the efficient-support portable constant-negative-pressure suction device for outdoor use provided in embodiments of the present disclosure;

FIG. 13 is a schematic perspective view showing a structure of the negative-pressure suction tube with no internal rifling, of the efficient-support portable constant-negative-pressure suction device for outdoor use provided in embodiments of the present disclosure, when sucking fluid at a negative pressure;

FIG. 14 is a schematic perspective diagram showing a structure of the negative-pressure suction tube with internal riflings of the efficient-support portable constant-negative-pressure suction device for outdoor use provided in embodiments of the present disclosure, when sucking fluid at a negative pressure;

FIG. 15 is a schematic perspective view showing a structure of internal groove lines of a suction control hole of the negative-pressure suction tube of the efficient-support portable constant-negative-pressure suction device for outdoor use provided in embodiments of the present disclosure;

FIG. 16 is a schematic perspective view showing a structure of the efficient-support portable constant-negative-pressure suction device for outdoor use provided in embodiments of the present disclosure, when used to suck sputum and blood in respiratory tract; and

FIG. 17 is a schematic perspective view showing a structure of the efficient-support portable constant-negative-pressure suction device for outdoor use provided in embodiments of the present disclosure, when used to suck contents in esophagus and stomach.

In the drawings: 2816—negative-pressure suction ball; 2815—circular gas exhaust valve; 2818—negative-pressure suction tube; 2817—suction control channel; 15—longitudinal elastic plate; 16—lateral elastic wire; 17—gas exhaust valve core; 19—gas exhaust valve spring; 18—gas exhaust valve hole; 13—suction control hole; 14—anti-backflow valve; 12—suction tube; 28151—negative-pressure gas exhaust valve; 21—drain hole; 20—manual drain valve; 22—support spring; 23—internal rifling; 24—internal groove line.

### DETAILED DESCRIPTION OF EMBODIMENTS

Below the technical solutions of the efficient-support portable constant-negative-pressure suction device for outdoor use of the present disclosure will be described clearly and completely in conjunction with drawings. Apparently, some but not all of examples of the present disclosure are described. Based on the examples of the present disclosure, all the other examples, obtained by a person ordinarily skilled in the art without paying inventive efforts, fall within the protection scope of the present disclosure.

In the description of the present disclosure, it should be noted that orientational or positional relationships indicated by terms such as “central”, “upper”, “lower”, “left”, “right”, “vertical”, “horizontal”, “inner” and “outer” are based on orientational or positional relationships as shown in the drawings, merely for facilitating describing the present disclosure and simplifying the description, rather than indicating or implying that related devices or elements have to be in the specific orientation, or to be configured or operated in specific orientation, therefore, they should not be construed as limiting the present disclosure. Besides, terms such as “first”, “second” and “third” are merely for distinguishing description, but should not be construed as indicating or implying importance in relativity.

In the description of the efficient-support portable constant-negative-pressure suction device for outdoor use of the present disclosure, it should also be noted that unless otherwise explicitly specified and defined, terms “mount”, “link” and “connect” should be construed in a broad sense. For example, the connection may be fixed connection, detachable connection, or integrated connection; it may be mechanical connection; it may also be electrical connection; it may be direct linking or indirect linking through an intermediate medium; and it may also be inner communication between two elements. For a person ordinary skilled in the art, specific meanings of the above-mentioned terms in the present disclosure can be understood according to specific circumstances.

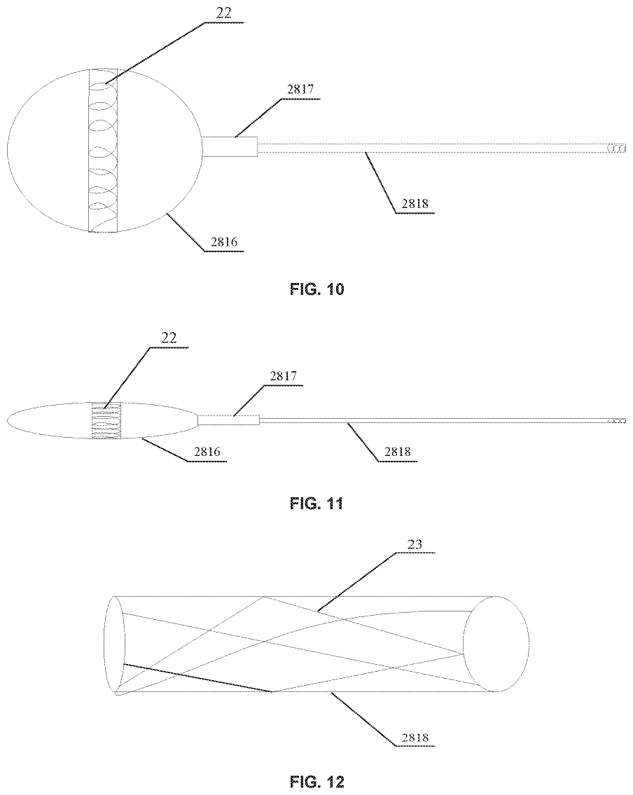

As shown in FIG. 1-FIG. 15, embodiments of the present disclosure provide an efficient-support portable constant-negative-pressure suction device for outdoor use, including: a negative-pressure suction ball 2816 extending from a proximal end to a distal end, wherein the negative-pressure suction ball 2816 is in an internal cystic structure, and the internal cystic structure includes a circular gas exhaust valve 2815, a balloon and an anti-backflow valve 14, which are sequentially arranged from the proximal end to the distal end; the device further includes a negative-pressure suction tube 2818, wherein negative-pressure suction tube 2818 communicates with the negative-pressure suction ball 2816, and the negative-pressure suction tube 2818 has a suction control channel 2817.

Compared with the prior art, the efficient-support portable constant-negative-pressure suction device for outdoor use of the embodiments of the present disclosure has following advantages:

the efficient-support portable constant-negative-pressure suction device for outdoor use provided in the embodiments of the present disclosure, when used outside hospitals, especially when used for rescuing the wounded in the field and battlefield or in places away from negative-pressure suction areas, can effectively solve the problems of airway obstruction caused by airway sputum and blood of the wounded and the sick, and especially prevent dyspnea, suffocation, hypoxia and even life-threatening cases of patients occurring due to chest puncture injury-caused bleeding or drowning-caused emergency airway conditions. Moreover, the efficient-support portable constant-negative-pressure suction device for outdoor use provided in the embodiments of the present disclosure has extremely convenient operation, and is simple, practical, scientific and safe, which can be used by both professional or non-professional persons after basic training, and can be also prepared for family elderly, which can effectively fill the huge gaps in the demand of future battlefield and market.

It should be additionally indicated here that regarding the basic research of the physical rheology that generates negative pressure: Bernoulli's equation: p+ρgh+(½)*ρv²=c, where p, ρ, v indicate the intensity of pressure, density and speed of a fluid, respectively; h indicates a vertical height; g indicates acceleration of gravity; and c indicates a constant. The Bernoulli's equation is an equation that reflects the relationship among speed, intensity of pressure, and other parameters in the movement of an ideal fluid. When an ideal positive-pressure fluid makes a steady motion under the action of a potential volume force, the movement equation (i.e., the Euler equation) is integrated along the streamline to obtain an equation expressing conservation of mechanical energy of a moving fluid. Negative-pressure suction also follows Bernoulli's equation and movement equation. The set sputum density ρ is maximum, h=0, the Bernoulli's equation can be simplified to be: p+(½)*ρv²=c, a minimum sectional area of an internal channel of the negative-pressure suction tube, which maintains a constant negative pressure, of the efficient-support portable constant-negative-pressure suction device for outdoor use defined by calculating according to the movement equation, however since the density of sputum is not constant in the test, castor oil is used as the basis standard for experimental research, wherein the viscosity of the castor oil is relatively high, and similar to the viscosity of the sputum and blood.

When the pressure change of the efficient-support portable constant-negative-pressure suction device for outdoor use is not too large, assuming that the flow of gas whose density can be viewed as a constant is used as a basic standard for stability research, it can be obtained by setting energy equations for two sections in the pipeline: P1−P2=(λL/r)V²/(2g);

The flow rate can be obtained from: V=√[2gr (P1−P2)/λL];
The flux: Q=(πr²/4) V=(πr²/4)*√[2gr (P1−P2)/λL];

In the equation: V: flow rate in the pipeline; Q: flux in the pipeline; g: acceleration of the gravity; r: inner diameter of the pipeline; P1: intensity of pressure at the starting end of the pipeline; P2: intensity of pressure at the tail end of the pipeline; λ: resistance coefficient of the pipeline wall; L: length of the pipeline. By calculating r, the inner diameter of the pipeline, and at the same time, negative pressure is generated when changing the negative-pressure suction ball, to satisfy the change of intensity of pressure inside the pipeline, satisfy generation of the pressure difference P1−P2, i.e. satisfy suction flow rate per unit time, and r, the inner diameter of the pipeline is one of the bases of the constant negative pressure of the efficient-support portable constant-negative-pressure suction device for outdoor use.

Regarding the study of materials science and material structure, when performing research on a constant-negative-pressure suction device convenient for field operation, materials science requires that when the elastic restoring force meets the use requirement, the negative pressure range may be −0.3099 kgf/cm² to −0.702 kgf/cm², single-person continuous manual operation is mainly adopted, the weight is in the range of 100-300 g, one-time molding is used, and no air leakage is achieved. The negative-pressure suction ball is set to be elliptical, with a maximal width of 55.87 mm and a minimal width is 21.64 mm.

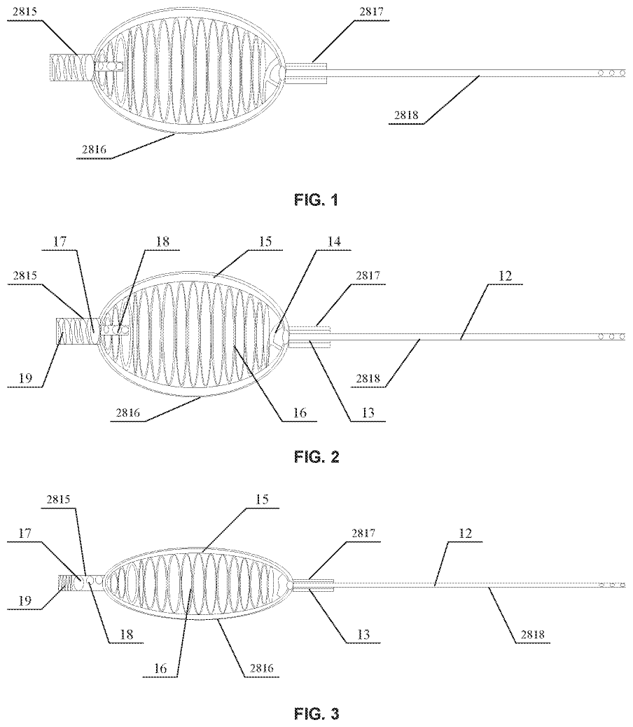

In the above, as shown in FIG. 1 and FIG. 2, the inner wall of the balloon of the above negative-pressure suction ball 2816 may be provided with a support member (not shown in the figures). The support member has an elastic restoring force and has property of resisting the squeezing of the balloon wall towards the center line of the negative-pressure suction ball 2816 to return to the spherical state.

The inner wall of the balloon of the negative-pressure suction ball 2816 may be also provided with a longitudinal elastic plate 15 and a lateral elastic wire 16, and the support member, together with the longitudinal elastic plate 15 and the lateral elastic wire 16, resists the squeezing of the balloon wall towards the center line of the negative-pressure suction ball 2816 to return to the spherical state.

Specifically, as shown in FIG. 1 and FIG. 2, the support member on the inner wall of the balloon of the above negative-pressure suction ball 2816 is in a soft structure, and the longitudinal elastic plate 15 and the lateral elastic wire 16 are fitted into the inner wall of the balloon of the negative-pressure suction ball 2816, wherein the longitudinal elastic plate 15 includes at least two ridge plates, and the lateral elastic wire 16 includes at least two lateral elastic wires which are alternate in pairs and combined with the longitudinal elastic plate 15.

The lateral elastic wire 16 may comprises multiple groups of lateral elastic wires. Each group has two lateral elastic wires 16, and two ends of each lateral elastic wire 16 are respectively connected to the ridge plates of the longitudinal elastic plate 15 which are arranged opposite to each other.

Optionally, the longitudinal elastic plate 15, the lateral elastic wire 16 and the balloon wall constitute an internal cystic structure of the negative-pressure suction ball 2816, and are formed by a one-time molding manufacturing process, which can effectively ensure that the elastic restoring force can form sufficient negative pressure in the balloon.

The circular gas exhaust valve 2815 may be tightly fitted to the proximal end of the inner balloon of the negative-pressure suction ball 2816, and the circular gas exhaust valve 2815 is composed of a gas exhaust valve core 17, a gas exhaust valve spring 19, and a gas exhaust valve hole 18. The circular gas exhaust valve 2815 is fabricated by a precision manufacturing process technology. The position where the negative-pressure suction ball 2816 is connected with the suction control hole 13 is integrated with an anti-backflow valve 14, wherein the anti-backflow valve 14 is fan-shaped, and a fan edge is fixed by a chordae tendineae, so as to facilitating squeezing and gas exhausting during use, and the anti-backflow valve 14 may prevent backflowing phenomenon of the sucked sputum and blood. In addition, the suction tube 12 is a hose and is formed by one-time sealing with the suction control hole 13.

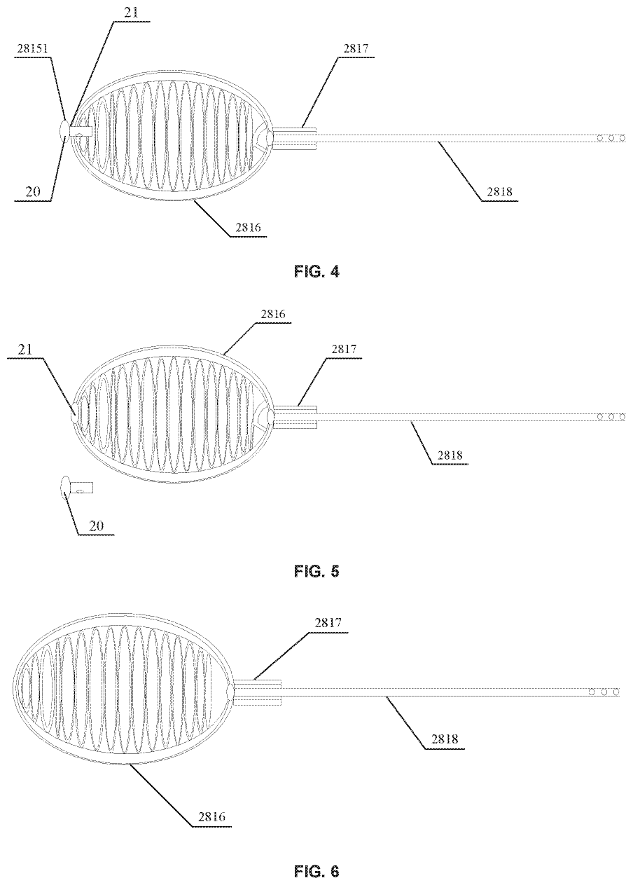

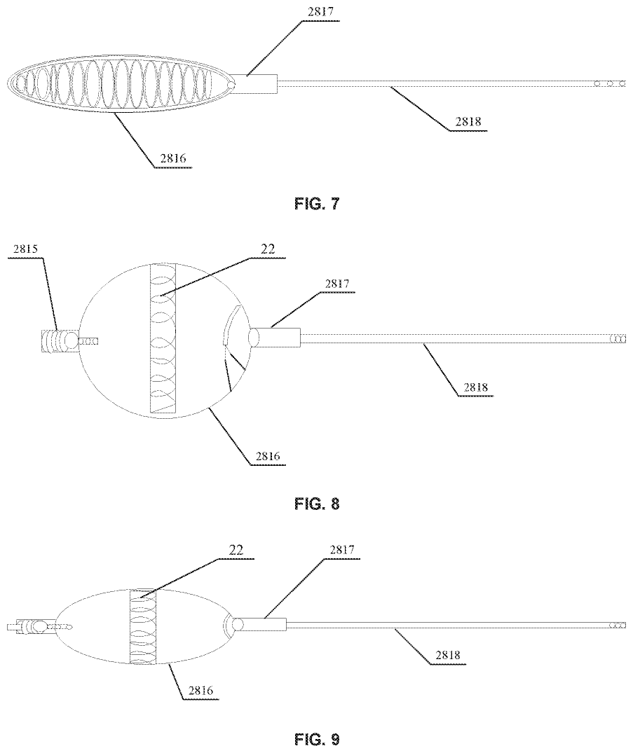

Optionally, as shown in FIG. 3, when the balloon of the negative-pressure suction ball 2816 is squeezed, the circular gas exhaust valve 2815 protrudes to exhaust gas, and the anti-backflow valve 14 closes the negative-pressure suction tube 2818; when the squeeze is released, the circular gas exhaust valve 2815 is retracted, and the anti-backflow valve 14 is opened; and when the balloon of the negative-pressure suction ball 2816 is in an expanded state, the balloon of the negative-pressure suction ball 2816 has a circular section and is elliptical as a whole. In addition, when the negative-pressure suction ball 2816 is squeezed, the gas exhaust valve core 17 of the circular gas exhaust valve 2815 compresses the gas exhaust valve spring 19, gas is discharged from the gas exhaust valve hole 18, and the anti-backflow valve 14 blocks the suction hole, so as to make the negative-pressure suction ball 2816 form a negative pressure.

As shown in FIG. 1-FIG. 15, embodiments of the present disclosure also provide an efficient-support portable constant-negative-pressure suction device for outdoor use, including: a movable negative-pressure gas exhaust valve 28151 extending from a proximal end to a distal end, a negative-pressure suction ball 2816 and a negative-pressure suction tube 2818, wherein the negative-pressure gas exhaust valve 28151 and the negative-pressure suction ball 2816 communicate with the negative-pressure suction tube 2818 through a suction control channel 2817. The negative-pressure suction tube 2818 may be integrally formed by injection molding or other molding processes.

Optionally, as shown in FIG. 4 and FIG. 5, the above negative-pressure gas exhaust valve 28151 has a manual knob, and the negative-pressure gas exhaust valve 28151 is provided with a knob port channel and a drain hole 21, wherein the manual knob is provided within the knob port channel and the drain hole 21, and can rotate in the knob port channel, and the manual knob can be taken out from the knob port channel and the drain hole 21, that is, the manual knob can close and open the drain hole. In addition, the negative-pressure suction ball 2816 may function as a gas exhaust valve hole, based on the negative-pressure gas exhaust valve 28151, and the manual drain valve 20 can also be used as the drain hole 21 to drain sputum or blood.

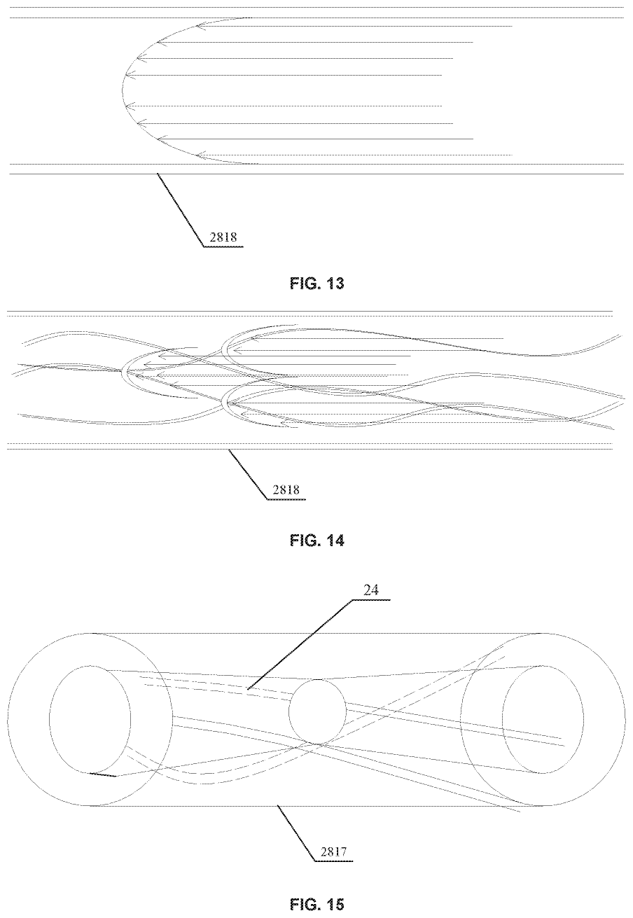

Optionally, as shown in FIG. 13 and FIG. 14, the balloon of the above negative-pressure suction ball 2816 has a balloon cavity, and the balloon cavity communicates with a negative-pressure oxygen suction tube.

Optionally, as shown in FIG. 8-FIG. 11, a support spring 22 is provided inside the balloon of the above negative-pressure suction ball 2816, and the support spring 22 is arranged laterally, and has two ends respectively connected to the inner wall of the balloon, such that when laterally squeezing the balloon, the support spring 22 can generate a restoring force to restore the shape of the balloon, thereby ensuring that the negative-pressure suction ball 2816 can form an effective negative pressure during the application process, and can be manufactured by injection molding or other molding processes; or a support spring 22 and an anti-backflow valve 14 are provided inside the balloon of the above negative-pressure suction ball 2816, wherein the anti-backflow valve 14 is provided at the place where the negative-pressure suction ball 2816 communicates with the suction control channel 2817, so as to prevent backflowing phenomenon of sucked sputum and blood.

Optionally, as shown in FIG. 12-FIG. 14, the inner tube wall of the above negative-pressure suction tube 2818 is provided with at least one internal rifling 23. The depth of the internal rifling 23 may be 0.1-0.3 mm, and the rifling is used to change the linear flow according to the hydrodynamics of the fluid head of the sputum or blood, and reduce the frictional resistance of the fluid during the flow line movement in the tube, to form tube flows not interfering with each other. Such rifling may be manufactured by injection molding or other molding processes and integrally formed at one time. In addition, according to the principles of hydrodynamics, although the sputum or blood has a relatively high viscosity and is not an ideal fluid, the flowing thereof in the negative-pressure suction tube 2818 also complies with basic hydrodynamics equations such as Bernoulli's equation.

Optionally, as shown in FIG. 15, the above negative-pressure suction tube 2818 has a suction control hole 13, and the hole wall of the suction control hole 13 is provided with at least one internal groove line 24. The internal groove line 24 of the suction control hole 13 has a depth of 0.2-0.5 mm and a width of 0.2-0.5 mm, and is used to reduce, according to the hydrodynamics of the fluid head of the sputum or blood, frictional resistance of the fluid during the flow line movement in the tube. Such groove line may be manufactured by injection molding or other molding processes and can be integrally formed at one time.

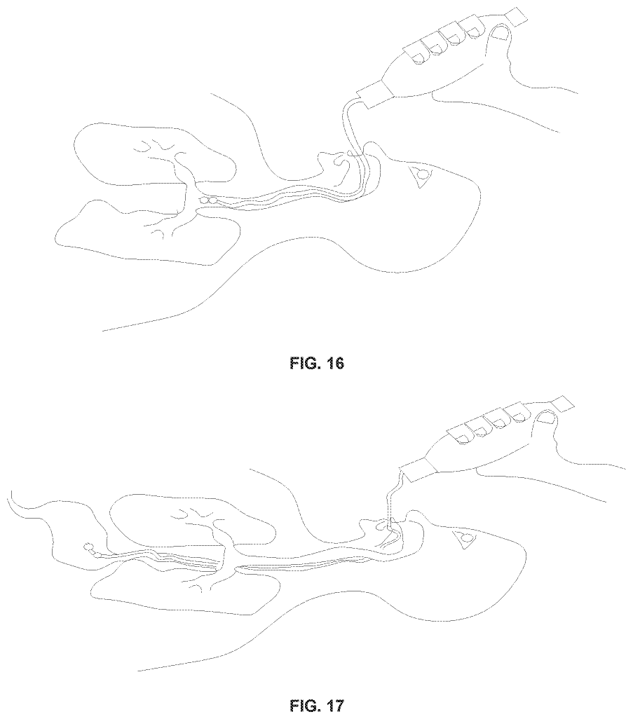

As shown in FIG. 16, when sucking sputum and blood, the efficient-support portable constant-negative-pressure suction device for outdoor use must have negative-pressure suction force which overcomes the viscosity of the sputum and blood and the resistance coefficient of the tube wall. In order to be suitable for field first aid, battlefield rescuing, home first aid and public occasions and be able to be opened and used within 30 seconds in an emergency, the negative-pressure suction ball 2816 generating the negative-pressure suction force has to be subjected to a tension elastic recovery research. For cases where there is much sputum and blood, the efficient-support portable constant-negative-pressure suction device for outdoor use can meet the requirements of continuous suction, without continuously and repeatedly pulling out and inserting the suction tube for sucking the sputum and blood, enabling simple and convenient operation.

As shown in FIG. 17, when sucking contents in esophagus and stomach, the efficient-support portable constant-negative-pressure suction device for outdoor use must have negative-pressure suction force which overcomes the viscosity of the contents in esophagus and stomach and the resistance coefficient of the tube wall. In order to be suitable for field first aid, battlefield rescuing, home first aid and public occasions and be able to be opened and used within 30 seconds in an emergency, the negative-pressure suction ball 2816 generating the negative pressure suction force has to be subjected to a tension elastic recovery research. For cases where there are many contents in esophagus and stomach, the efficient-support portable constant-negative-pressure suction device for outdoor use can meet the requirements of continuous suction, without continuously and repeatedly pulling out and inserting the suction tube for sucking the contents in esophagus and stomach, enabling simple and convenient operation.

The efficient-support portable constant-negative-pressure suction device for outdoor use provided by the embodiments of the present disclosure, which is a tool for both military and civilian purposes, is a suction tool suitable for rescuing the wounded and the sick whose respiratory tracts have much sputum and bleeding or whose buccal, esophageal or gastric secretions need to be urgently handled on site, in the field, outdoor informal medical places, battlefields or emergency accident places; and relates to a geometric structure modification of disposable medical rescue tool designed and researched based on material mechanics, fluid mechanics and aerodynamics.

The above are only preferred embodiments of the present disclosure and are not intended to limit the present disclosure. Any modification, equivalent replacement, improvement, etc. made within the spirit and principle of the present disclosure should fall within the scope of protection of the present disclosure.

### INDUSTRIAL APPLICABILITY

The efficient-support portable constant-negative-pressure suction device for outdoor use provided in the present disclosure is a tool for both military and civilian purpose; is suitable for rescuing the wounded and the sick whose respiratory tracts have much sputum and bleeding or whose buccal, esophageal or gastric secretions need to be urgently handled on site, in the field, outdoor informal medical places, battlefields or emergency accident places; and has a wide application prospect in the field of first aid, and has the advantage of mass production.

1. An efficient-support portable constant-negative-pressure suction device for outdoor use, comprising:
a negative-pressure suction ball extending from a proximal end to a distal end, wherein the negative-pressure suction ball is in an internal cystic structure, and the internal cystic structure comprises a circular gas exhaust valve, a balloon and an anti-backflow valve, which are sequentially arranged from the proximal end to the distal end,
further comprising: a negative-pressure suction tube, wherein the negative-pressure suction tube communicates with the negative-pressure suction ball, and the negative-pressure suction tube has a suction control channel.

2. The efficient-support portable constant-negative-pressure suction device for outdoor use according to claim 1, wherein a support member is provided on an inner wall of the balloon of the negative-pressure suction ball, wherein the support member is configured to resist squeezing of a balloon wall of the balloon toward a center line of the negative-pressure suction ball, to make the balloon return to a spherical state.

3. The efficient-support portable constant-negative-pressure suction device for outdoor use according to claim 2, wherein the support member on the inner wall of the balloon of the negative-pressure suction ball is in a soft structure.

4. The efficient-support portable constant-negative-pressure suction device for outdoor use according to claim 2, wherein a longitudinal elastic plate and a lateral elastic wire are provided on the inner wall of the balloon of the negative-pressure suction ball,
wherein the longitudinal elastic plate and the lateral elastic wire in combination with the support member together resist squeezing of the balloon wall of the balloon toward the center line of the negative-pressure suction ball, to make the balloon return to the spherical state.

5. The efficient-support portable constant-negative-pressure suction device for outdoor use according to claim 4, wherein the longitudinal elastic plate and the lateral elastic wire are fitted in the negative-pressure suction ball, and the longitudinal elastic plate comprises at least two ridge plates which are provided opposite to each other, and at least two lateral elastic wires are provided, wherein two ends of each of the at least two lateral elastic wires are connected to two opposite ridge plates, respectively, such that each of the at least two lateral elastic wires is cooperated with the longitudinal elastic plate.

6. The efficient-support portable constant-negative-pressure suction device for outdoor use according to claim 1, wherein when the balloon of the negative-pressure suction ball is squeezed, the circular gas exhaust valve protrudes to exhaust gas, and the anti-backflow valve closes the negative-pressure suction tube; and when the squeezing is released, the circular gas exhaust valve is retracted, and the anti-backflow valve is opened.

7. The efficient-support portable constant-negative-pressure suction device for outdoor use according to claim 1, wherein when the balloon of the negative-pressure suction ball is squeezed to be in an expanded state, the balloon of the negative-pressure suction ball has a circular section and is elliptical as a whole.

8. The efficient-support portable constant-negative-pressure suction device for outdoor use according to claim 1, wherein the circular gas exhaust valve comprises a gas exhaust valve core, a gas exhaust valve spring and a gas exhaust valve hole, wherein the gas exhaust valve spring, the gas exhaust valve core and the gas exhaust valve hole are sequentially provided from the proximal end to the distal end of the efficient-support portable constant-negative-pressure suction device for outdoor use.

9. The efficient-support portable constant-negative-pressure suction device for outdoor use according to claim 1, wherein the anti-backflow valve is fan-shaped, and a fan edge of the fan-shaped anti-backflow valve is fixed by a chordae tendineae.

10. An efficient-support portable constant-negative-pressure suction device for outdoor use, comprising: a movable negative-pressure gas exhaust valve extending from a proximal end to a distal end of the efficient-support portable constant-negative-pressure suction device for outdoor use, a negative-pressure suction ball and a negative-pressure suction tube, wherein the negative-pressure gas exhaust valve and the negative-pressure suction ball communicate with the negative-pressure suction tube through a suction control channel.

11. The efficient-support portable constant-negative-pressure suction device for outdoor use according to claim 10, wherein the negative-pressure gas exhaust valve has a manual knob, and the negative-pressure gas exhaust valve is provided with a knob port channel and a drain hole which communicate sequentially to each other, wherein the knob port channel communicates with a balloon of the negative-pressure suction ball, the manual knob is provided inside the knob port channel and the drain hole, and the manual knob is configured to be taken out from the knob port channel and the drain hole.

12. The efficient-support portable constant-negative-pressure suction device for outdoor use according to claim 10, wherein the balloon of the negative-pressure suction ball has a balloon cavity, and the balloon cavity communicates with a negative-pressure oxygen suction tube.

13. The efficient-support portable constant-negative-pressure suction device for outdoor use according to claim 10, wherein a support spring is provided inside the balloon of the negative-pressure suction ball, wherein the support spring is laterally arranged to enable the support spring to resist squeezing of a balloon wall of the balloon toward a center line of the negative-pressure suction ball, to make the balloon return to a spherical state.

14. The efficient-support portable constant-negative-pressure suction device for outdoor use according to claim 10,
wherein a support spring and an anti-backflow valve are provided inside the balloon of the negative-pressure suction ball, wherein the support spring is laterally arranged to enable the support spring to resist squeezing of a balloon wall of the balloon toward a center line of the negative-pressure suction ball, to make the balloon return to a spherical state, and the anti-backflow valve is provided at a position where the negative-pressure suction ball communicates with the suction control channel.

15. The efficient-support portable constant-negative-pressure suction device for outdoor use according to claim 10, wherein an inner tube wall of the negative-pressure suction tube is provided with at least one internal rifling.

16. The efficient-support portable constant-negative-pressure suction device for outdoor use according to claim 15, wherein the at least one internal rifling has a depth of 0.1-0.3 mm.

17. The efficient-support portable constant-negative-pressure suction device for outdoor use according to claim 10, wherein the negative-pressure suction tube has a suction control hole, and a hole wall of the suction control hole is provided with at least one internal groove line.

18. The efficient-support portable constant-negative-pressure suction device for outdoor use according to claim 17, wherein the at least one internal groove line of the suction control hole has a depth of 0.2-0.5 mm and a width of 0.2-0.5 mm.

19. The efficient-support portable constant-negative-pressure suction device for outdoor use according to claim 2, wherein when the balloon of the negative-pressure suction ball is squeezed, the circular gas exhaust valve protrudes to exhaust gas, and the anti-backflow valve closes the negative-pressure suction tube; and when the squeezing is released, the circular gas exhaust valve is retracted, and the anti-backflow valve is opened.

20. The efficient-support portable constant-negative-pressure suction device for outdoor use according to claim 2, wherein when the balloon of the negative-pressure suction ball is squeezed to be in an expanded state, the balloon of the negative-pressure suction ball has a circular section and is elliptical as a whole.
